(19) United States
(12) Patent Application Publication
(10) Pub. No.: US 2023/0244356 A1
(43) Pub. Date: Aug. 3, 2023

(54) TIME INTENSITY DOMINANCE OF SENSATION (TIDS)

(71) Applicant: Cargill, Incorporated, Wayzata, MN (US)

(72) Inventors: Brian GUTHRIE, Chanhassen, MN (US); Nicolas Jean Yves GUILBOT, Breda (NL)

(73) Assignee: Cargill, Incorporated, Wayzata, MN (US)

(21) Appl. No.: 18/000,159

(22) PCT Filed: May 6, 2021
(86) PCT No.: PCT/US2021/031073

§ 371 Date: Nov. 29, 2022

Related U.S. Application Data
(60) Provisional application No. 63/032,293, filed on May 29, 2020.

(51) Int. Cl. G06F 3/0482

(52) U.S. Cl. CPC G06F 3/0482

(57) ABSTRACT

The subject matter herein includes apparatus and techniques, such as can be used to automatically assess dominance of sensation. For example, a technique for such assessment can include generating a representation of two or more sensations for display to a user, receiving respective selections of respective sensations amongst the two or more sensations in response to display of the representation. As an example, receiving the respective selections of respective sensations can include obtaining data indicative of a magnitude of the respective sensations corresponding to the respective selections and obtaining data indicative of a temporal relationship between the respective selections, providing time-intensity and dominance of sensation data contemporaneously.

### CROSS-REFERENCE TO RELATED APPLICATIONS

This application claims the benefit of U.S. Provisional Application No. 63/032,293, filed May 29, 2020, and entitled “TIME INTENSITY DOMINANCE OF SENSATION (TIDS),” which is incorporated by referenced herein in its entirety.

### FIELD OF THE DISCLOSURE

This document pertains generally, but not by way of limitation, to characterizing dominance of sensation during tasting of a sample, and more particularly to tools for quantifying and tracking an intensity of a dominant sensation versus time while a panelist tastes a sample.

### BACKGROUND

Researchers have developed a variety of techniques to attempt to reproducibly and reliably capture data from panelists concerning sensory perceptions, particularly in relation to sensory perceptions concerning food attributes. Sensory perception profiling can be used by food scientists to better understand how various ingredients, combinations of ingredients, or processing may drive perceptible attributes or changes in such perceptible attributes. Data concerning such perceptions may also provide indications correlating with a like or dislike of particular products by panelists. Use of sensory profiling techniques can assist researchers in objectively comparing candidate product ingredient formulations or processing techniques. Such evaluation can also be used for many other purposes, such as to help develop or refine new products, to help define specifications, or to provide process monitoring or control. As an illustrative example, sensory perception profiling can be used to characterize an impact of a substitute ingredient or formulation change on perceptible attributes from the perspective of test panelists, such as to assist in one or more of validating, developing, or modifying a product or ingredient.

### SUMMARY OF THE DISCLOSURE

As mentioned above, various techniques can be used to capture data relating to sensory perception, and particularly in relation to sensory perception concerning food attributes. Such attributes can be grouped generally into categories such as involving one or more of taste, aroma, or texture, as examples, and such attributes can be referred to as sensations. As an illustrative example, in one approach, a temporal dominance of sensation (TDS) technique can be used, such as to obtain data indicative of a dominant attribute versus time from the perspective of a panelist. In another approach, a panelist can be asked to “check all attributes that apply” and selections of the panelist can be tracked versus time. In the above two examples (TDS and check-all-that-apply), no data indicative of a magnitude of a particular attribute is obtained. In yet another approach, a panelist can provide an indication of an intensity of a single selected attribute over time (e.g., a time-intensity evaluation technique). In the time-intensity example, no data indicative of a dominance of one attribute versus another is provided. Generally, the techniques above (TDS, check-all-that-apply, and time-intensity) can be referred to as dynamic sensory profiling techniques, in the sense that such techniques may be used to track a perception that changes in some way over time.

The present inventors have recognized, among other things, that a sensory perception testing framework can be provided that allows panelists to generate data indicative of both time-intensity and dominance of sensation versus time, such as using a graphical user interface (GUI) that captures such time-intensity and dominance data contemporaneously. Such contemporaneous capture of time-intensity and dominance sensory perception data can be referred to as a “Time-intensity Dominance of Sensation” (TiDS) technique. The present inventors have also recognized, among other things, that providing a user interface and related processing techniques as described herein can solve a technical challenge of transforming indicia of panelist sensory perception, such as can include categorical data, into functional data, in a repeatable and intuitive manner facilitating one or more of analysis, visualization, or reporting. The techniques described herein can also be used to evaluate a quality of the resulting data, such as to identify intra-panelist or inter-panelist sources of variation. Generally, the techniques described herein can also be used to generate various reports and visualizations of acquired data, including transformations or analysis such as parameterization, component analysis, or analysis of variance by panelist, by attribute, or by combination of attributes, as illustrative examples.

In an example, a technique such as a computer-implemented or otherwise machine-implement method can be used for automatically assessing dominance of sensation, technique comprising generating a representation of two or more sensations for display to a user, receiving respective selections of respective sensations amongst the two or more sensations in response to display of the representation, wherein the receiving the respective selections of respective sensations comprises, obtaining data indicative of a magnitude of the respective sensations corresponding to the respective selections, and obtaining data indicative of a temporal relationship between the respective selections.

For example, the obtaining data indicative of a magnitude of the respective sensations can include obtaining a location of an indicium from a user placed within a selectable region corresponding to a respective sensation amongst the two or more sensations. The indicium may be provided using at least one of a touch-sensitive surface or a mouse, and obtaining the data indicative of the magnitudes and temporal relationships corresponds to a trajectory of the indicium over time. Obtaining the data indicative of the magnitude can include determining a distance of the indicium from a reference location, such as where reference location comprises a central region of the representation.

This summary is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates generally an example comprising a system, such as can be used to perform one or more sensory perception assessment techniques as shown and described herein.

FIG. 2A shows an illustrative example such as an arrangement of regions that can be presented to a user (e.g., a panelist), with the regions corresponding to respective sensations for selection by the user, arranged about a central region.

FIG. 2B shows another illustrative example such as an arrangement of regions that can be presented to a user (e.g., a panelist), with the regions corresponding to respective sensations for selection by the user arranged radially from a central region of the representation.

FIG. 2C shows a trajectory defined by input from a user received over time overlaid on a presentation to a user (e.g., a graphical user interface).

FIG. 2D shows an illustrative example comprising a representation of time-intensity dominance of sensation (TiDS) corresponding to the trajectory in FIG. 2D.

FIG. 3 shows yet another illustrative example such as an arrangement of regions that can be presented to a user (e.g., a panelist), with the regions corresponding to respective sensations for selection by the user arranged radially from a central region of the representation, where the sensations concern sensory perceptions reported by the panelist in relation to the panelist tasting a substance-under-test.

FIG. 4 illustrates generally a technique, such as an automated method for obtaining time-intensity dominance of sensation (TiDS) data, such as using a presentation as shown illustratively in other examples herein.

FIG. 5 illustrates generally a technique, such as a workflow for one or more of transforming or presenting time-intensity dominance of sensation (TiDS) data, such as data obtained using a presentation as shown illustratively in other examples herein.

FIG. 6A shows an illustrative example of a visualization of time-intensity dominance of sensation (TiDS) data obtained using an automated technique.

FIG. 6B shows another illustrative example of a visualization of time-intensity dominance of sensation (TiDS) data obtained using an automated technique.

FIG. 7 shows yet another illustrative example of a visualization of time-intensity dominance of sensation (TiDS) data obtained using an automated technique.

FIG. 8A illustrates generally an example comprising two representations of time-intensity dominance of sensation (TiDS) data, such as corresponding to two separate assessments and including respective landmarks.

FIG. 8B illustrates generally an example comprising transforming the assessments of FIG. 8A to one or more of shift or scale such assessments in time to align a respective landmark in a first assessment with a corresponding landmark in a second assessment.

FIG. 9A shows an illustrative example comprising representations of time-intensity attribute data including respective landmarks, the plot shown in FIG. 9A obtained using an automated technique.

FIG. 9B shows an illustrative example comprising transforming the assessments of FIG. 9A to one or more of shift or scale such assessments in time to align a respective landmark in each assessment with a corresponding landmark in the other assessments.

FIG. 9C shows an illustrative example comprising a representation of landmarked assessments, such as representing various samples.

FIG. 10 shows an illustrative example comprising a phase-plot visualization of an attribute from sampled time-intensity dominance of sensation (TiDS) data.

FIG. 11 shows an illustrative example comprising a radar-plot visualization of significance levels (e.g., “p-values”) resulting from analysis samples, such as can be obtained using an analysis of variance (ANOVA).

FIG. 12A and FIG. 12B shows illustrative examples comprising plots of two components or “harmonics” that can be extracted using a principal component analysis, and in FIG. 12B, a corresponding time-domain representation of a contribution or weighting of one of the harmonics of FIG. 12A.

FIG. 13 illustrates a block diagram of an example comprising a machine upon which any one or more of the techniques (e.g., methodologies) discussed herein may be performed.

### DETAILED DESCRIPTION

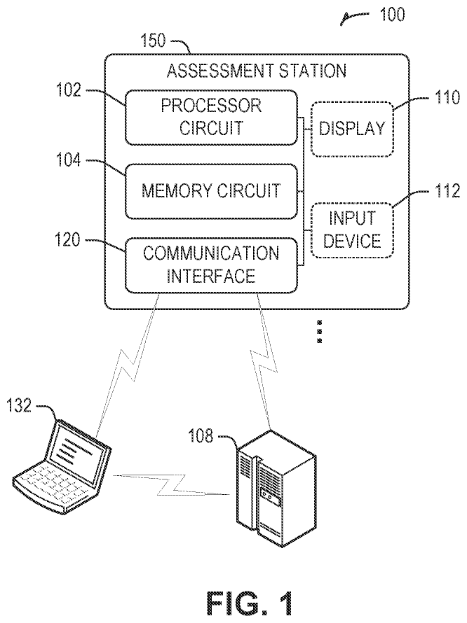

As mentioned above, the present inventor has recognized, among other things, that a problem exists in performing sensory perception assessments where data is obtained that is indicative of a dominance of a particular attribute amongst two or more attributes, along with an intensity of perception of the dominant attribute, versus time. To address such a challenge, the present inventor has developed apparatus and techniques, such as can include use of a graphical user interface to provide a presentation for a user, such as a test panelist, to facilitate obtaining such sensory perception data. The present inventor has also, among other things, developed a flexible workflow that can be used to establish test protocols, and to transform and analyze resulting data indicative of sensory perception obtained via execution of the test protocols. Sensory perception assessment and related analytical techniques described herein can be computer-implemented or otherwise machine-implemented. For example, FIG. 1 illustrates generally an example comprising a system 100, such as can be used to perform one or more assessment techniques as shown and described herein. In the example of FIG. 1, different elements in the system 100 can be used to implement a time-intensity dominance of sensation (TiDS) sensory perception assessment. For example, an assessment station 150 (such as a desktop, laptop, tablet, or mobile device) can execute an instance of an assessment routine or can be used to present a routine instantiated using a server 108 (e.g., a world-wide-web server) or other resource, such as according to a specified test protocol.

The assessment station can include at least one processor circuit 102, and at least one memory circuit 104 that can store instructions that, when executed by the processor circuit 102, cause the processor circuit 102 to present a graphical representation using a display 110, and to receive user input using at least one input device 112. For example, the assessment station 150 can include one or more input devices such as a keypad, a keyboard, a mouse, a touchscreen or other digitizer, or another input device. Results of assessments can be stored either locally using the assessment station 150, or in an on-site or off-site repository such as a centralized server or network-attached storage resource (e.g., provisioned through a cloud storage provider). For example, the assessment station 150 can include a wired or wireless communication interface 120, such as to communicate with a server 108 or other resource. One or more assessment stations 150 can be located at a test site, and execution of various tests may be coordinated using an on-site controller 132 such as another desktop, laptop, tablet, or mobile device. Additionally, or alternatively, a virtual test site can be established, and distributed assessment stations at different locations can be configured from a remote location, such as to establish a coordination capability for multi-site assessment or otherwise provide for centralized coordination of assessments.

A researcher may use an on-site controller or another device to retrieve results from the server 108 or from another resource. For example, analytical techniques such as corresponding to workflows or data visualizations described herein can be triggered, manipulated, or otherwise instantiated using the controller 132 or another device. Numerically intensive evaluation need not be performed using the controller 132 and may instead be requested and a server 108 or other cloud resource can be provisioned, such as on an as-needed basis, to perform numerical analysis, and results can be transmitted back to the controller 132 or another client, or stored for further evaluation such as in a queued manner. Security protocols can be established and automatically enforced, such as to prevent users (e.g., panelists) operating an assessment station from manipulating results or accessing test or other panelist data unless appropriate permissions have been associated with the user. In a similar manner, the assessment station 150 can be programmed to execute a specified test protocol, such as prompting a panelist to take specific actions or facilitating execution of specified sequence of assessments, such as can include replicates or variant assessments according to a desired experiment design.

As an illustrative example, a user interface that can be instantiated at either the controller 132 or the assessment station 150 (or both) can include tools to let a test administrator build test protocols such as assigning panelists (or requesting assignment of panelists for blind trials), selecting facilities to be used for testing, selecting language preferences or other localization, and the like. Similarly, panelists can be presented with prompts to login or otherwise provide credentials authenticating the panelist. Such authentication can be logged to provide audit capabilities relating to panelist or administrator activities.

The assessment station 150 may be equipped to provide auditory feedback, either in relation to a TiDS assessment during the test or in order to prompt a user orally concerning instructions. For example, as shown and described elsewhere herein, auditory feedback can be provided during testing including an audible indication occurring contemporaneously with receiving a respective selection of a respective sensation (e.g., sensory attribute) and the audible indication can vary relative to a selection. In this manner, the user (e.g., a panelist) can receive either visual or audible feedback, or both, relating to an intensity of a sensation in order to facilitate receiving an indication of such sensation from the user.

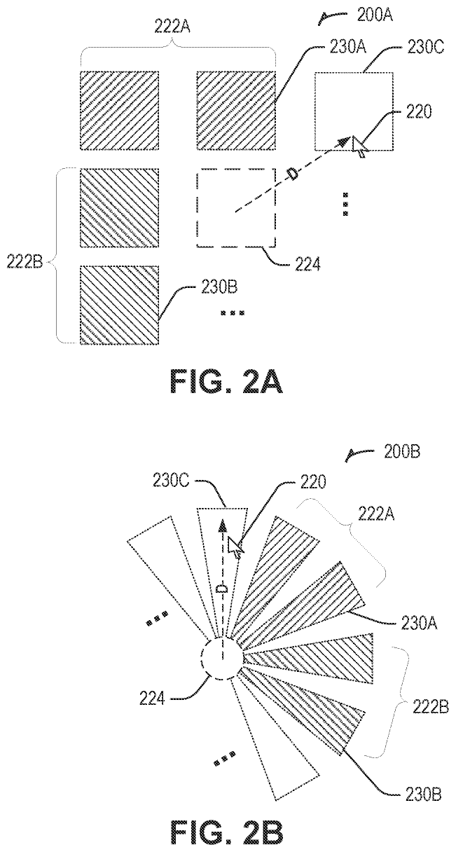

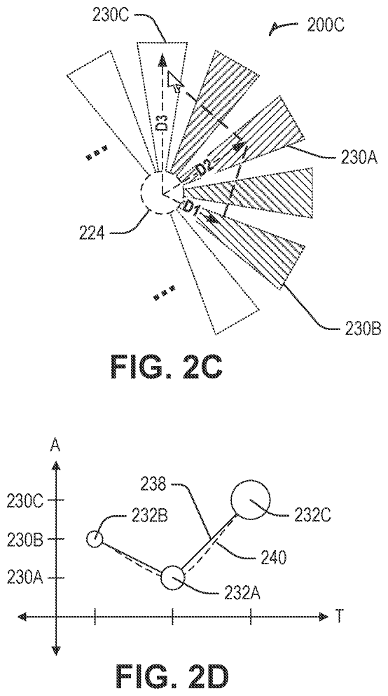

FIG. 2A shows an illustrative example such as an arrangement 200A of regions that can be presented to a user (e.g., a panelist) using an automated assessment system such as via an assessment station 150 as shown in FIG. 1. In FIG. 2A, the regions corresponding to respective sensations (e.g., attributes) can be selected by the user and can be arranged about a central region 224. For example, groups of related sensations such as a first group 222A including a first sensation corresponding to a region 230A and a second group 222B including a second sensation corresponding to a second region 230B can be located about a central region 224. A selection, such as performed using a mouse or touchpad pointer 220 or received via a digitizer (e.g., touchscreen) can be performed by a panelist, such as to indicate both a species and an intensity of sensation. The species of sensation can correspond to the selected region (e.g., regions 230A, 230B, and 230C can each correspond to a different sensation). Such sensations can generally be referred to as attributes, and in the context of assessment of taste, texture, or aroma, such attributes can include descriptors such as “creamy,” “thick,” chunky” (e.g., texture descriptors); or “sweet,” “salty,” “sour” (e.g., taste descriptors), as illustrative (but non-restrictive) examples. The central region 224 can be used to trigger user interface events, such as starting or stopping an assessment in response to selection (e.g., clicking or touching) of the central region 224 or another region by the user.

Intensity data can be obtained such as by capturing data indicative of a distance, “D,” of the selection location (e.g., indicated by pointer 220) from a specified reference location. The reference location in FIG. 2A can be defined as the center of the central region 224. In this manner, a species (e.g., a particular attribute indicated by a user selecting a particular region 230A, 230B, or 230C, for example) and an intensity can be indicated contemporaneously. Other presentations can be used.

For example, FIG. 2B shows another illustrative example such as an arrangement 200B of regions that can be presented to a user (e.g., a panelist), with the regions corresponding to respective sensations for selection by the user arranged radially from a central region 224 of the representation. The arrangement 200B of FIG. 2B provides a visual cue to a panelist that as a radius from the central region 224 increases, a recorded intensity value will also increase, because the regions taper from a narrow width at the central region 224 to a wider width at the periphery. As in the example of FIG. 2A, in FIG. 2B, groups of related sensations (e.g., attributes) can be clustered nearby each other, such as first group 222A including a first sensation 230A, and a second group 222B including a second sensation 230B. Also, in a manner similar to FIG. 2A, a pointer 220 location or other indicium can be received from a user, and a distance, “D” of the indicium from a reference location can be used to obtain data indicative of the intensity of the selected sensation (e.g., in the illustrative example of FIG. 2B, the sensation corresponding to the region 230C is selected).

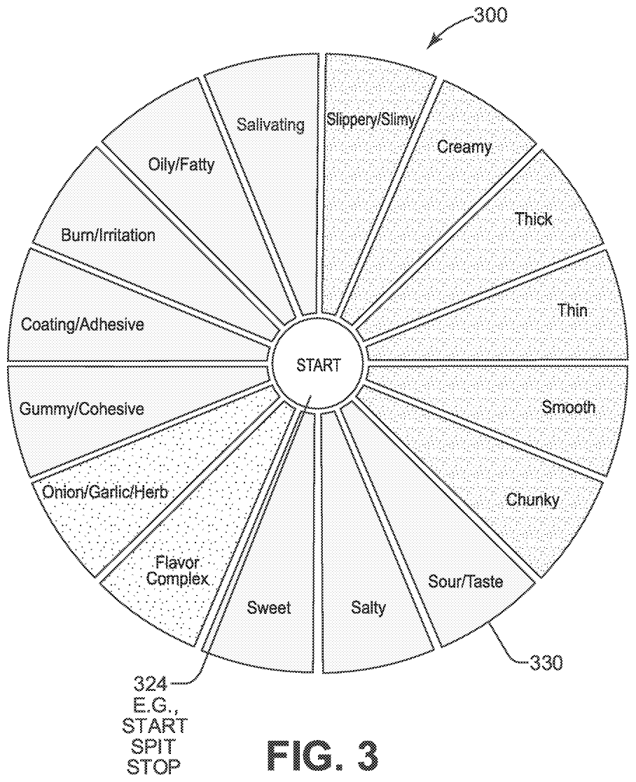

The present inventor has recognized, among other things, that a presentation such as shown illustratively in the examples of FIG. 2A, FIG. 2B, and FIG. 3 can be used to obtain data indicative of both a selected dominant sensation and its intensity as perceived by the user, including obtaining data indicative of a temporal relationship between respective selections, such as during contemporaneous sampling of a product or ingredient by the user (e.g., selections are made as the user is tasting the product or just after the user spits out the product). For example, FIG. 2C shows a trajectory defined by input from a user received over time overlaid on a presentation to a user (e.g., a graphical user interface). In the example of FIG. 2C, when prompted by a user interface event (such as a display to commence selecting), or in response to a user selection (such as triggering a user interface event), a trajectory of selections can be sampled over time, such as providing a sequence of respective selections over time.

For example, as shown in FIG. 2C, a user might select (e.g., touch a touch-sensitive display or select using a mouse pointer) a central region 224 to start an assessment, such as just before or while the user is tasting a substance-under-test, and the user can then move an indicium such as a pointer or otherwise select a sequence of locations as the user's perception of the substance evolves over time. In FIG. 2C, inputting a trajectory can include selecting a sensation 230B at a first intensity defined by a distance, “D1,” then at a later time, selecting another sensation 230A at a second intensity corresponding to a distance, “D2,” and then selecting yet another sensation 230C at a third intensity corresponding to a distance, “D3.” During or after such a sequence, the user may provide other input, such as selecting the central region 224 to indicate either that the user spit out the substance-under-test or indicating an end of a test, for example. Such start, spit, or stop events can also be logged including logging temporal data indicative of a relationship of such events with other selections (e.g., temporally relating user interface events such as selections of the central region with other data indicative of either dominance or intensity or both).

FIG. 2D shows an illustrative example comprising a representation of time-intensity dominance of sensation (TiDS) corresponding to the trajectory in FIG. 2D. In the example of FIG. 2D, a plot can be presented to a user (e.g., a researcher or analyst), such as contemporaneously displaying a representation of both a dominant sensation, corresponding to a categorical attribute location along the vertical axis (e.g., indicating one of sensations corresponding to regions 230A, 230B, or 230C) versus time along the horizontal axis. An intensity of such a selected sensation can also be indicated such as by one or more of a a size, a shape, or a color of a location (e.g., a point or locus) along the plot, such as shown illustratively in FIG. 2D. For example, a first sample 232B can have a symbol size or diameter that is smaller than corresponding symbols for a second sample 232A and a third sample 232C. A simple piece-wise linear curve 238 can be defined either graphically or analytically for further functional analysis, or a spline or other technique can be applied such as to provide a curve 240. For example, similarly, plots can be generated for respective sensations, and a linear or spline-based representation can be established to provide functional analysis capability on an attribute-by-attribute basis. For example, because the data shown in FIG. 2D is categorical (e.g., showing sensations as attributes along the vertical axis), an additive smoothing technique can be used to facilitate functional analysis either of a representation of a dominance plot as shown in FIG. 2D, or on an attribute-by-attribute basis. A quality of curve-fitting or smoothing can be evaluated numerically or even graphically. For example, a lambda plot showing GCV value (e.g., a “score”) versus lambda values can be generated to assist in assessing a goodness of a smoothing operation, such as when an additive smoothing technique is used.

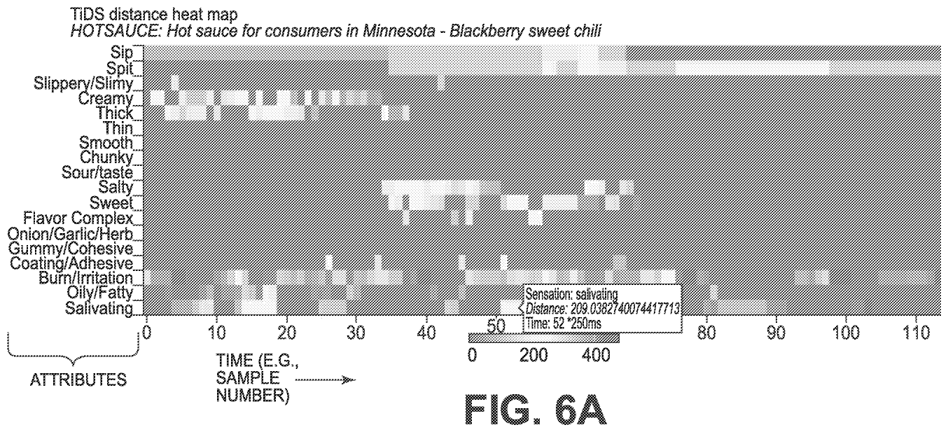

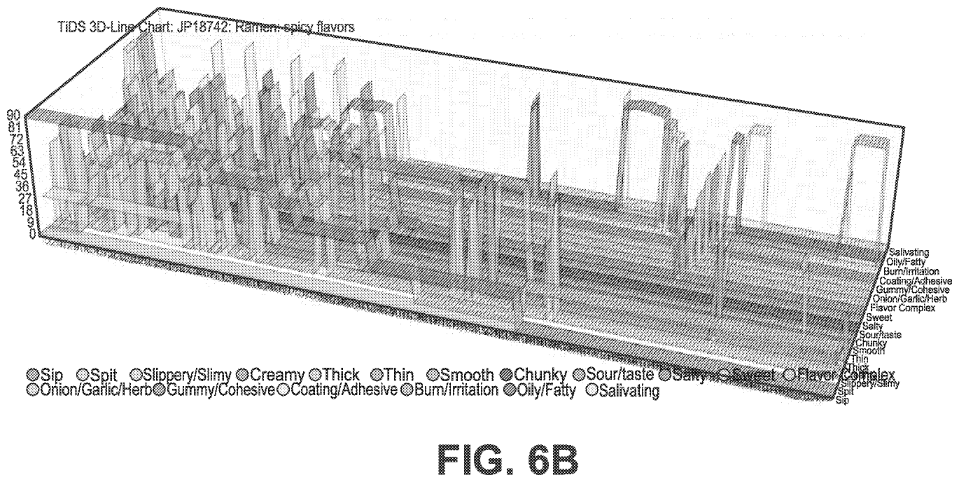

In the example of FIG. 2D, and other examples herein, category samples or intensity values may be acquired using a discrete-time acquisition scheme, such as having a specified interval between acquisitions. For example, as shown in FIG. 6, the interval between successive acquisitions can be 250 milliseconds, corresponding to an acquisition rate of 4 samples per second.

FIG. 3 shows yet another illustrative example such as an arrangement 300 of regions that can be presented to a user (e.g., a panelist), with the regions corresponding to respective sensations for selection by the user arranged radially from a central region of the representation, where the sensations concern sensory perceptions reported by the panelist in relation to the panelist tasting a substance-under-test. In a manner similar to the examples of FIG. 2A, FIG. 2B, and FIG. 2C, a user (e.g., a panelist) can select a region labeled with a sensation (e.g., corresponding to a perceived attribute) that most closely describes a sensation experienced by the user, contemporaneously. Different sensations can be grouped together and a visual indication of such grouping can be provided, such as by providing regions having similar shading or coloring (shown illustratively as different shades of gray in FIG. 3). Each region, such as a selectable region 330 can include a label describing the sensation, and a central region 324 can be used for triggering various events, such as for receiving a selection from a user to start sampling, or to provide an indication to a user that sampling has started, or that the user has spit out a substance-under-test, or that testing is to be stopped.

As in the other examples herein, a distance of a location of a user selection can be determined from a reference location, and such a distance can provide an indication of an intensity of the perceived sensation from the perspective of the user. Audible feedback can be provided contemporaneously with selection, such as varying in one or more of frequency or magnitude as an indicium of selection (e.g., a mouse pointer location or locus touched by a user on a touch-screen display) moves radially outward from the central region 324. For example, a tone can be sounded having a frequency or range of frequencies specified to fall within a range of hearing of a broad age range of users, such as to facilitate audible feedback even for users having diminished high-frequency hearing. The tone can become relatively louder in response to a selection by the user having a relatively greater distance from the central region, and the tone can become relatively quieter in response to a selection by the user having a relatively lesser distance from the central region. As an illustrative example, an audio frequency of about 8 kilohertz (KHz) or some other audible frequency can be used to provide the audible feedback.

The sensations and corresponding selectable regions shown in FIG. 3 can include sensations that have been experimentally validated in some manner, such as established through trials using reference test substances or in a manner conforming to a published standard or scientific literature, as illustrative examples. A user, such as a panelist, may receive prior training materials or perform practice assessments using the arrangement 300 to familiarize the user with the available sensations and their spatial locations relative to each other. In this manner, the user can be prepared to rapidly select an appropriate sensation during “actual” assessment (as opposed to “training”) without having to search for a corresponding sensation.

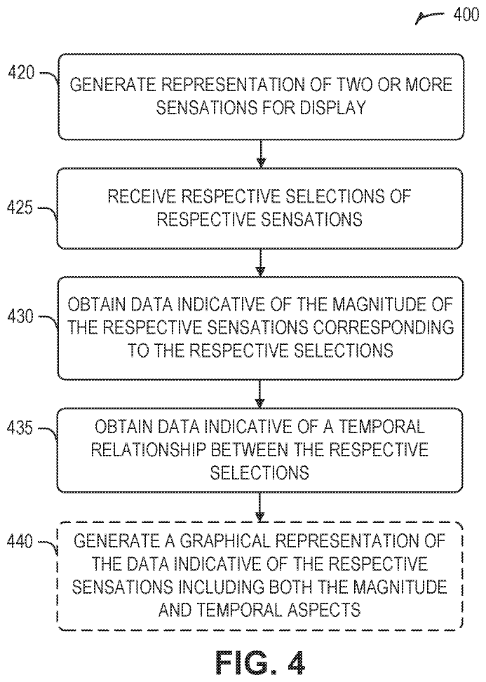

FIG. 4 illustrates generally a technique 400, such as an automated method for obtaining time-intensity dominance of sensation (TiDS) data, such as using a presentation as shown illustratively in other examples herein. At 420, the technique 400 can include generating a representation of two or more sensations for display to a user. For example, such a representation can include a visual arrangement similar to one or more of the examples of FIG. 2A, FIG. 2B, or FIG. 3, as illustrative examples. At 425, respective selections of respective sensations can be received from the user. Such selections can be discrete selections such as the user clicking on respective regions or touching respective regions, or the selections can include a continuous swipe or drag operation, such as across two or more different regions corresponding to different sensations. At 430, data indicative of a magnitude of respective selected sensations can be obtained. Such data can be derived from a location of an indicium provided by the user, such as a location where the user touched a touchscreen or placed a mouse pointer to perform selection. As discussed above, in one approach, intensity values can be established using a distance metric, but such an approach is not the only technique that may be used. Other techniques can include tactile techniques, such as responding a pressure or force provided by the user or using another input such as a ribbon, slider, or rotary control arrangement, either using physical ribbon, slider, or rotary controls, or presenting representations thereof on a graphical user interface for manipulation by a user.

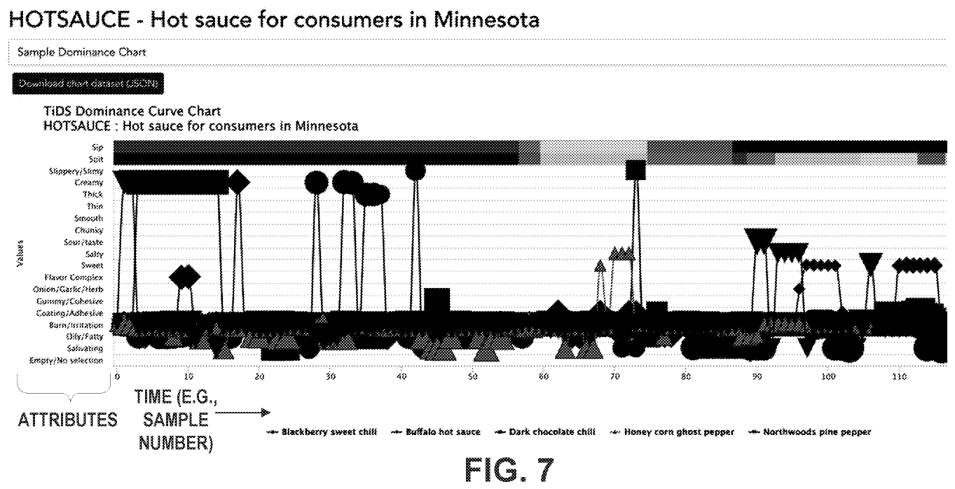

At 435, data indicative of a temporal relationship between the respective selections can be obtained. Such temporal relationships can be established by using a sample-based acquisition scheme where a time-domain series (e.g., a time-series) is generated corresponding to successive acquisitions, where an acquisition includes a sample index or timestamp, a value corresponding to a selected attribute as a categorical variable, and a value corresponding to an intensity of the selected attribute. In this manner, a time-domain record is established preserving the temporal relationship between respective selections. Optionally, at 440 a graphical representation such as a plot or heat-map of data indicative of the respective sensations can be generated, such as including both the magnitude and temporal aspects. Examples of such representations are shown in FIG. 2D, FIG. 6A, FIG. 6B, and FIG. 7.

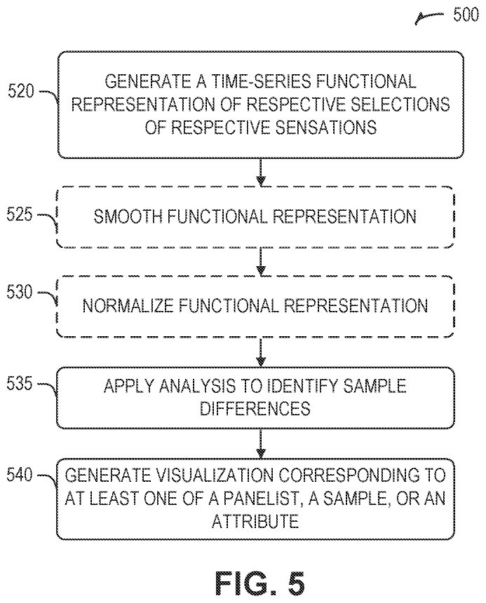

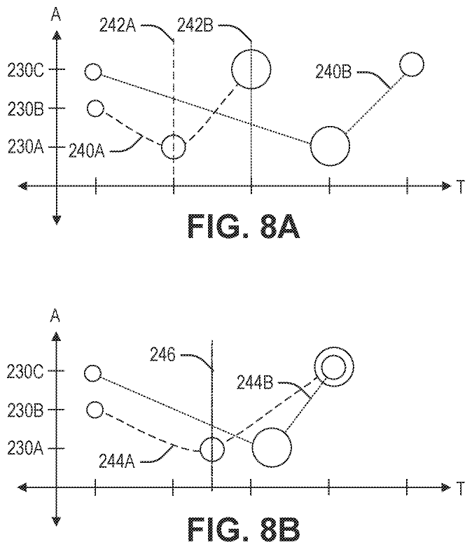

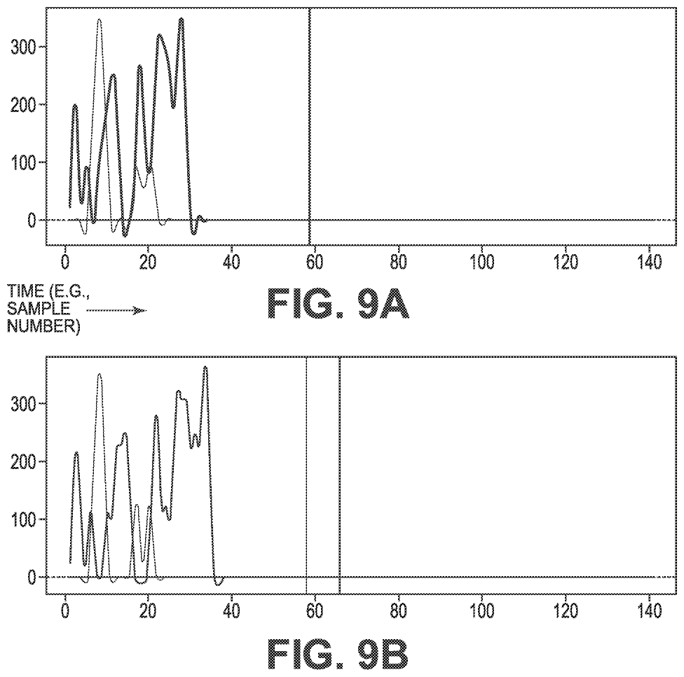

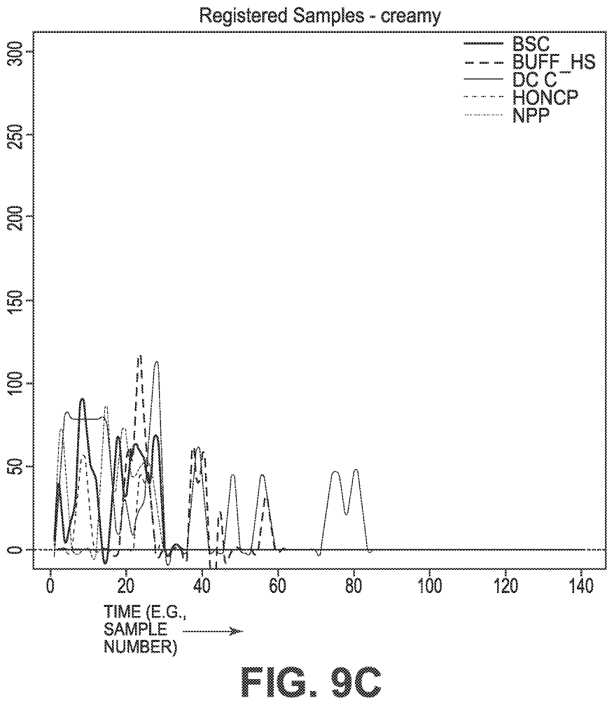

FIG. 5 illustrates generally a technique 500, such as a workflow for one or more of transforming or presenting time-intensity dominance of sensation (TiDS) data, such as data obtained using a presentation as shown illustratively in other examples herein. As mentioned above, a time-series representation can be generated, corresponding to respective selection of respective sensations received from a user, such as at 520. The time-series representation of a particular assessment can be defined as a family of time-series representations where each attribute has a corresponding time-series indicating an intensity of the respective attribute versus time, or the time-series representation can include assigning numerical values to categorical sensation selections. A functional representation can be established such as by applying a spline or performing a regression on the time-series. The time-series data can be smoothed, optionally, at 525, such as using an additive smoothing technique. Optionally, at 530, the functional representation can be normalized. Some analytical operations can be performed using un-normalized data, such as analytical evaluation to determine panelist reproducibility or sensitivity. Normalization can include scaling or shifting acquired data in time, such as to align respective landmarks between samples as shown and described in relation to FIG. 8A, FIG. 8B, FIG. 9, FIG. 9B, and FIG. 9C. Referring to FIG. 5, at 535, analysis can be applied to identify sample differences between respective assessments, such as including one or more of analysis of variance, component analysis (e.g., principal component analysis or individual component analysis as a blind-source selection approach to de-noise samples), or t-testing. Such analysis can be used to assess variation or reproducibility by panelist, by assessment, or by attribute, or in relation to other variables. At 540, a visualization can be generated corresponding to at least one of a panelist, a sample (e.g., an assessment instance corresponding to a specified product or ingredient), or an attribute (e.g., a sensation).

The workflow of FIG. 5 is illustrative and other workflows can be used. For example, a workflow can include observing panelist performance or training level, such as computing analytical values relating to panelist sensitivity or discrimination (e.g., intra-panelist variation) in view of repeated trials rating a sample or group of samples, such as a reference sample. Panelist performance can also be assessed against other panelists, such as to evaluate consensus or otherwise identify inter-panelist variation. A report can be generated, such as providing coefficients rating a panelist's ability to reliably discriminate between attributes for a given sample across multiple trials or in comparison to a reference profile defining an ideal panelist or representing an aggregated representation of panelists (such as a reference profile defined as a median or other central tendency of similar data obtained from other panelists).

FIG. 6A shows an illustrative example of a visualization of time-intensity dominance of sensation (TiDS) data obtained using an automated technique. In FIG. 6A, a “heat map” view is provided, where categorical attributes corresponding to selected sensations are shown on the vertical axis, along with events such as sipping or spitting out a substance being evaluation, and the horizontal axis is representative of time (e.g., an acquisition index or “sample index”). Intensity values can be represented by tone or color, such as shown in FIG. 6A illustratively. The visualization of FIG. 6A can represent an assessment performed by a single panelist, with the tone or color indicative of intensity values corresponding to respective selections by the panelist. In another example, a visualization similar to FIG. 6A can include an aggregation of assessments, either from a single panelist or multiple panelists. Such aggregation can include a sum of intensity values at each time, or a central tendency such as an average or median intensity, as illustrative examples. In this manner, the visualization of FIG. 6A can be used to visualize dominance and intensity across panelists for a given sample, or each plot in the chart can represent a central tendency of a sample so that dominance can be viewed across several samples. As an illustrative example, each plot in FIG. 6A can represent a different sample, where the values and dominant attributes represent a median computed across panelists for each time step.

FIG. 6B shows another illustrative example of a visualization of time-intensity dominance of sensation (TiDS) data obtained using an automated technique. Similar to the example of FIG. 6A, FIG. 6B presents the values as extending orthogonally from plane to provide a three-dimensional line-curve representation, by contrast to the heat map visualization of FIG. 6A.

FIG. 7 shows another illustrative example of a visualization of time-intensity dominance of sensation (TiDS) data obtained using an automated technique. The visualization in FIG. 7 can represent data in a manner similar to FIG. 6A or FIG. 6B, but instead of showing a “heat map” or 3D line curves, intensity values can be represented in manner similar to FIG. 2D, where a size of a marker at respective locations in the plot corresponds to a sampled intensity value. The visualization of FIG. 7 permits multiple TiDS plots to be shown contemporaneously, such as by using one or more of different colors, tones, or marker shapes for each plot. Events such as sipping or spitting out a substance-under-test can also be shown, such as in manner similar to FIG. 6A, such as at the top of the plot.

As mentioned above, and elsewhere herein, a landmarking technique can be used to help reduce sample variation between panelists or between assessments performed by a panelist. For example, use of such a landmarking approach can help to remove individual panelist effects when data is aggregated. FIG. 8A illustrates generally an example comprising two representations of time-intensity dominance of sensation (TiDS) data, such as corresponding to two separate assessments and including respective landmarks. The arrangement of the plots shown in FIG. 8A and FIG. 8B is generally similar to FIG. 2D. A first assessment 240A can be defined by respective selections of respective sensations, and an event 242A, such as sensations and events logged during selection of sensations by a user when presented with a graphical user interface as shown and described elsewhere herein. The event 242A, for example, may indicate spitting out a substance under test, as an illustration. A second assessment 240B can similarly include selected sensations and an event 242B.

In FIG. 8B, the assessments 240A and 240B of FIG. 8A can be transformed to one or more of shift or scale such assessments in time to align a respective landmark in a first assessment with a corresponding landmark in a second assessment. For example, events occurring at time indices denoted by the lines at 242A and 242B can be used as landmarks, such as assigned to a specified time index or sample index in FIG. 8B, and the assessment 240A can be stretched to provide a landmarked assessment 244A as shown in FIG. 8B. Similarly, the assessment 240B can be compressed in time to provide the landmarked assessment 244B. A location for aligned landmark 246 can be arbitrary or can be established using temporal indices of the landmarks 242A and 242B. For example, an average, median, or other central tendency of temporal indices of landmarks 242A and 242B can be used to establish the aligned landmark 246. Once the assessments have been landmarked or otherwise normalized as shown in FIG. 8B (or FIG. 9B, for example), further analysis can be performed on the assessments or a functional representation thereof

FIG. 9A shows an illustrative example comprising representations of time-intensity attribute data including respective landmarks, the plot shown in FIG. 9A obtained using an automated technique. Each of the shaded curves in FIG. 9A can correspond to an assessment performed by a panelist, with the vertical axis representing intensity data corresponding to a particular attribute. Vertical lines can represent different non-aligned events such as spitting events. For example, FIG. 9A can show two different trials performed on the same type of sample by the same panelist. In FIG. 9B, the vertical lines have been time-aligned, such as by scaling the respective shaded curves in time (e.g., compressing or expanding the curves to establish a uniform record length where the events are assigned a specified time index).

FIG. 9C shows an illustrative example comprising a representation of landmarked assessments, such as representing various samples. In FIG. 9C, different samples have been landmarked in a manner similar to FIG. 9B. In this manner, time-aligned representations of TiDS data from different samples can be viewed, such as for a particular attribute in this example. By contrast, a family of plots similar to FIG. 9C can be generated showing different attributes for a single sample.

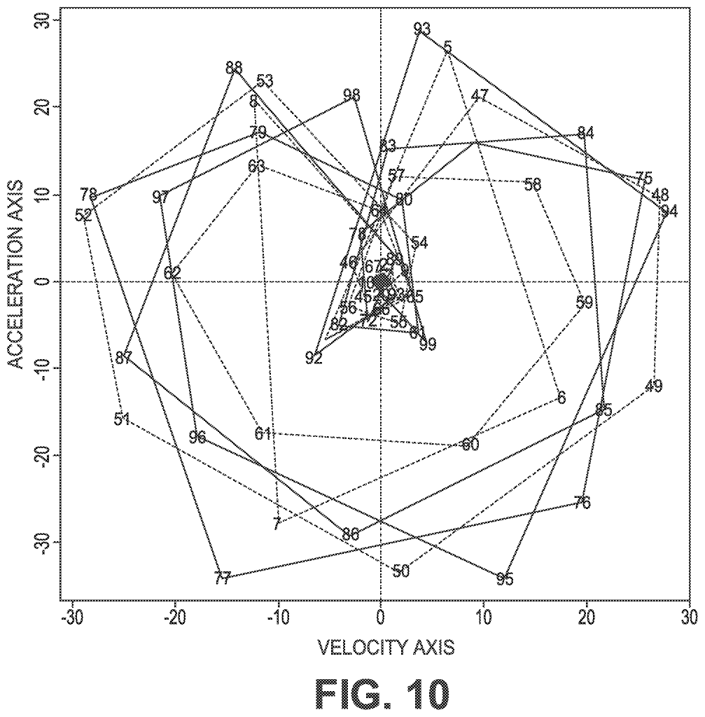

A variety of visualizations can be performed on assessment data, either on individual attributes within an assessment, or in relation to data aggregated from multiple assessments. For example, FIG. 10 shows an illustrative example comprising a phase-plot visualization of an attribute from sampled time-intensity dominance of sensation (TiDS) data. In the example of FIG. 10, a velocity axis and an acceleration axis are shown. Values for velocity and acceleration can be computed such as using a finite-difference technique applied to time-series data corresponding to intensity values of a particular attribute (e.g., selected sensation) over time. In this manner, first and second derivative values can be estimated from the time series. While the velocity and acceleration terminology refers to physical kinematic activity, such a phase plot may still provide a useful framework for analyzing and considering evolution of sensory perception, particularly in relation to panelists tasting substances-under-test. For example, the phase-plot representation can provide hints concerning potential versus kinetic aspects of flavor, texture, or aroma evolution in a dynamic sense. Many other visualizations or reports can be generated using a sampled time-intensity dominance of sensation (TiDS) assessment as described in this document.

Generally, the techniques described herein can also be used to generate various reports and visualizations of acquired data, including transformations or analysis such as parameterization, component analysis, or analysis of variance by panelist, by attribute, or by combination of attributes, as illustrative examples.

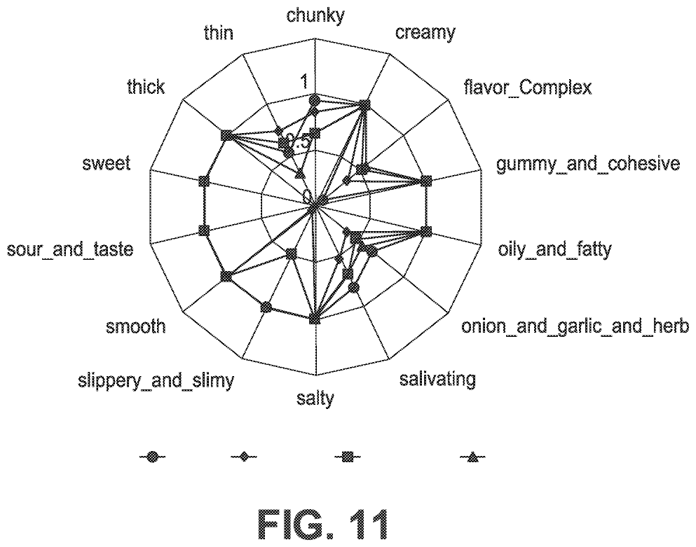

For example, FIG. 11 shows an illustrative example comprising a radar-plot visualization of significance levels (e.g., “p-values”) resulting from analysis samples, such as can be obtained using an analysis of variance (ANOVA). Generally, in FIG. 11, p-values lower in magnitude than a specified threshold can be considered statistically significant. In this manner, attributes showing low p-values (e.g., points towards the center of plot) across multiple samples indicate statistically significant attribute differences. The configuration of the radar plot could be inverted, having a magnitude of unity at the center and extending to zero or another specified threshold at the periphery. In such an example, attributes having points extending further outward from the center of the radar plot could indicate significance from a statistical perspective.

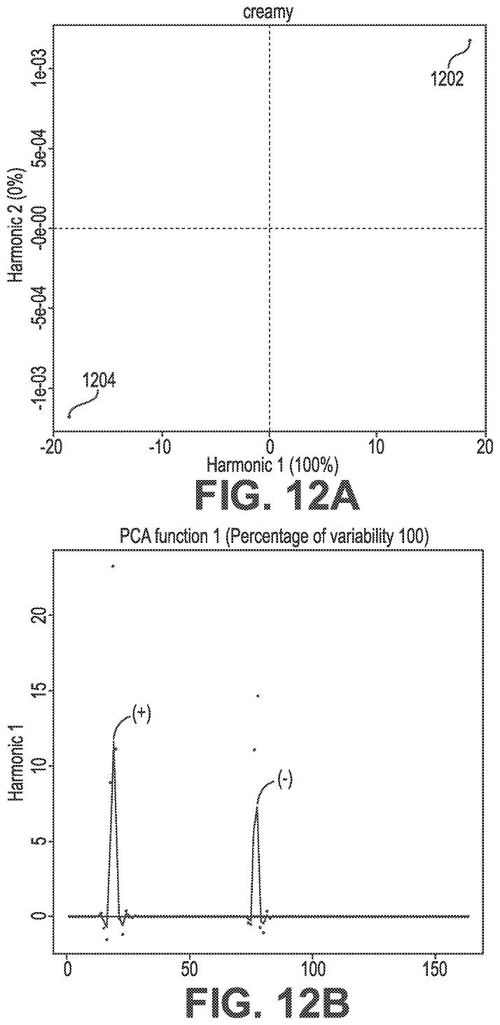

FIG. 12A and FIG. 12B shows illustrative examples comprising plots of two components or “harmonics” that can be extracted using a principal component analysis, and in FIG. 12B, a corresponding time-domain representation of a contribution or weighting of one of the harmonics of FIG. 12A. Use of principal component analysis across a family of panelists relative to a particular sample may reveal underlying or “latent” variables or “harmonics” that correlate with observed dominance patterns, intensity patterns, or both. The representations of FIG. 12A and FIG. 12B can assist in visualization of how harmonics contribute relative to each other, and versus time. For example, at location 1202, harmonic “1” from the horizontal axis indicates a positive contribution to the observed TiDS data. There is no way to detect when this contribution occurs from FIG. 12A. However, in FIG. 12B, the location marked (+) shows a duration where this contribution occurs temporally during sampling (e.g., where during tasting or other sampling the latent variable is expressed). Similarly, at location 1204, a negative contribution from harmonic “1” is observed, and in FIG. 12B, the location marked (−) shows that this negative weighting occurs later in time. Alternatively, or in addition, plots such as FIG. 12A or the phase plot of FIG. 10 could be generated for animated presentation, showing an evolution of points or other indicia over time in a manner corresponding to time progression of the sample.

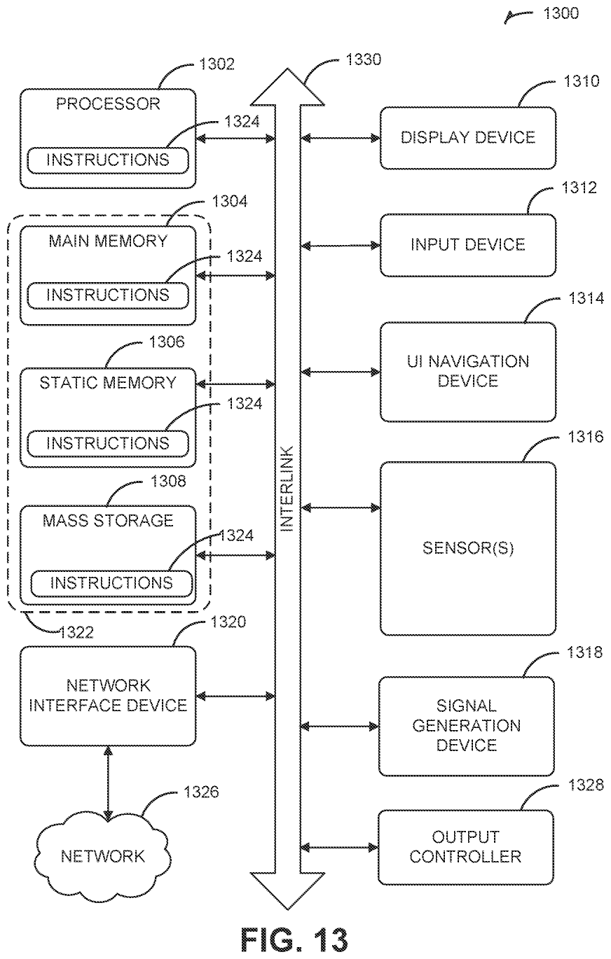

FIG. 13 illustrates a block diagram of an example comprising a machine 1300 upon which any one or more of the techniques (e.g., methodologies) discussed herein may be performed. In various examples, the machine 1300 may operate as a standalone device or may be connected (e.g., networked) to other machines. In a networked deployment, the machine 1300 may operate in the capacity of a server machine, a client machine, or both in server-client network environments. In an example, the machine 1300 may act as a peer machine in peer-to-peer (P2P) (or other distributed) network environment. The machine 1300 may be a personal computer (PC), a tablet device, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuitry is a collection of circuits implemented in tangible entities that include hardware (e.g., simple circuits, gates, logic, etc.). Circuitry membership may be flexible over time and underlying hardware variability. Circuitries include members that may, alone or in combination, perform specified operations when operating. In an example, hardware of the circuitry may be immutably designed to carry out a specific operation (e.g., hardwired). In an example, the hardware comprising the circuitry may include variably connected physical components (e.g., execution units, transistors, simple circuits, etc.) including a computer readable medium physically modified (e.g., magnetically, electrically, such as via a change in physical state or transformation of another physical characteristic, etc.) to encode instructions of the specific operation. In connecting the physical components, the underlying electrical properties of a hardware constituent may be changed, for example, from an insulating characteristic to a conductive characteristic or vice versa. The instructions enable embedded hardware (e.g., the execution units or a loading mechanism) to create members of the circuitry in hardware via the variable connections to carry out portions of the specific operation when in operation. Accordingly, the computer readable medium is communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time.

Machine (e.g., computer system) 1300 may include a hardware processor 1302 (e.g., a central processing unit (CPU), a graphics processing unit (GPU), a hardware processor core, or any combination thereof), a main memory 1304 and a static memory 1306, some or all of which may communicate with each other via an interlink (e.g., bus) 1308. The machine 1300 may further include a display unit 1310, an alphanumeric input device 1312 (e.g., a keyboard), and a user interface (UI) navigation device 1314 (e.g., a mouse). In an example, the display unit 1310, input device 1312 and UI navigation device 1314 may be a touch screen display. The machine 1300 may additionally include a storage device (e.g., drive unit) 1316, a signal generation device 1318 (e.g., a speaker), a network interface device 1320, and one or more sensors 1321, such as a global positioning system (GPS) sensor, compass, accelerometer, or other sensor. The machine 1300 may include an output controller 1328, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 1316 may include a machine readable medium 1322 on which is stored one or more sets of data structures or instructions 1324 (e.g., software) embodying or utilized by any one or more of the techniques or functions described herein. The instructions 1324 may also reside, completely or at least partially, within the main memory 1304, within static memory 1306, or within the hardware processor 1302 during execution thereof by the machine 1300. In an example, one or any combination of the hardware processor 1302, the main memory 1304, the static memory 1306, or the storage device 1316 may constitute machine readable media.

While the machine readable medium 1322 is illustrated as a single medium, the term “machine readable medium” may include a single medium or multiple media (e.g., a centralized or distributed database, and/or associated caches and servers) configured to store the one or more instructions 1324.

The term “machine readable medium” may include any medium that is capable of storing, encoding, or carrying instructions for execution by the machine 1300 and that cause the machine 1300 to perform any one or more of the techniques of the present disclosure, or that is capable of storing, encoding or carrying data structures used by or associated with such instructions. Non-limiting machine readable medium examples may include solid-state memories, and optical and magnetic media. Accordingly, machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic or other phase-change or state-change memory circuits; magnetic disks, such as internal hard disks and removable disks; magneto-optical disks; and CD-ROM and DVD-ROM disks.

The instructions 1324 may further be transmitted or received over a communications network 1326 using a transmission medium via the network interface device 1320 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks such as conforming to one or more standards such as a 4G standard or Long Term Evolution (LTE)), Plain Old Telephone (POTS) networks, and wireless data networks (e.g., Institute of Electrical and Electronics Engineers (IEEE) 802.11 family of standards known as Wi-Fi®, IEEE 802.15.4 family of standards, peer-to-peer (P2P) networks, among others. In an example, the network interface device 1320 may include one or more physical jacks (e.g., Ethernet, coaxial, or phone jacks) or one or more antennas to connect to the communications network 1326. In an example, the network interface device 1320 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1300, and includes digital or analog communications signals or other intangible medium to facilitate communication of such software.

Each of the non-limiting aspects above can stand on its own, or can be combined in various permutations or combinations with one or more of the other aspects or other subject matter described in this document.

The above detailed description includes references to the accompanying drawings, which form a part of the detailed description. The drawings show, by way of illustration, specific embodiments in which the invention can be practiced. These embodiments are also referred to generally as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A method of automatically assessing dominance of sensation, the method comprising:
generating a representation of two or more sensations for display to a user;
receiving respective selections of respective sensations amongst the two or more sensations in response to display of the representation, wherein the receiving the respective selections of respective sensations comprises:
obtaining data indicative of a magnitude of the respective sensations corresponding to the respective selections; and
obtaining data indicative of a temporal relationship between the respective selections.

2. The method of claim 1, wherein the representation of the two or more sensations includes a graphical representation comprising selectable regions corresponding to each sensation.

3. The method of claim 1, wherein the generating the representation comprises generating respective regions corresponding to each sensation, the regions extending radially from a central region of the representation.

4. The method of claim 1, wherein the obtaining data indicative of a magnitude of the respective sensations includes obtaining a location of an indicium from a user placed within a selectable region corresponding to a respective sensation amongst the two or more sensations.

5. The method of claim 4, wherein the indicium is provided using at least one of a touch-sensitive surface or a mouse, and wherein obtaining the data indicative of the magnitudes and temporal relationships corresponds to a trajectory of the indicium over time.

6. The method of claim 4, wherein the obtaining the data indicative of the magnitude includes determining a distance of the indicium from a reference location.

7. The method of claim 6, wherein the reference location comprises a central region of the representation.

8. The method of claim 4, wherein obtaining data indicative of the magnitude includes generating an audible indication of the location of the indicium, the generating the audible indication occurring contemporaneously with receiving a respective selection of a respective sensation and the audible indication varying relative to the location.

9. The method of claim 8, wherein the audible indication varies in at least one of magnitude or frequency in response to the location.

10. The method of claim 1, wherein the receiving respective selections of respective sensations amongst the two or more sensations commences in response to a first user interface event.

11. The method of claim 10, wherein the first event comprises receiving a first indication from the user to commence receiving respective selections of respective sensations during a first phase of a test.

12. The method of claim 10, wherein the method comprises receiving a second indication from the user corresponding to a second user interface event to commence receiving respective selections of respective sensations during a second phase of a test.

13. The method of claim 12, wherein the two or more sensations comprise taste sensations, wherein the first user interface event corresponds to an initiation of a taste test including a subject tasting a substance-under-test;
wherein the second user interface event corresponds to the subject spitting out the substance-under-test.

14. The method of claim 13, comprising obtaining two or more time-series representations of respective selections including magnitude data.

15. The method of claim 14, comprising scaling one or more respective time-series representations in time to align respective instants corresponding to a specified event.

16. The method of claim 14, comprising shifting one or more respective time-series representations in time to align respective instants corresponding to a specified event.

17. The method of claim 14, comprising smoothing the two or more time-series representations.

18. The method of claim 1 comprising generating a plot representing a magnitude over time of a selected sensation amongst the two or more sensations.

19. The method of claim 18, wherein the magnitude over time is represented using at least one of a color or luminance.

20. The method of claim 1, comprising generating a phase plot having at least two axes, using a selected time series corresponding to a selected sensation amongst the two or more sensations.

21. The method of claim 20, wherein a first axis amongst the at least two axes comprises a velocity axis and a second axis amongst the at least two axes comprises an acceleration axis.

22. The method of claim 21, comprising estimating respective velocities and accelerations using finite-difference techniques operating on the selected time series.

23. The method of claim 1, comprising assigning numerical values to respective sensations amongst the two or more sensations.

24. The method of claim 23, comprising generating a dominance time series by selecting a respective sensation meeting a specified criterion at corresponding instants in the dominance time series.

25. The method of claim 24, wherein the specified criterion corresponds to the respective sensation having the greatest magnitude at the corresponding instant in the dominance time series.

26. The method of claim 24, comprising smoothing the dominance time series.

27. A system comprising at least one processor circuit and at least one memory circuit, the memory circuit comprising executions that, when executed by the at least one processor circuit, cause a system to perform the method of claim 1.

28. The system of claim 27, further comprising a display to present the representation of two or more sensations for display to a user and an input device to receive respective selections of respective sensations amongst the two or more sensations in response to display of the representation.

29. A computer readable medium comprising instructions that, when executed by at least one processor circuit, cause a system to perform the method of claim 1.
